(19) United States
(12) Patent Application Publication
(10) Pub. No.: US 2015/0025280 A1
(43) Pub. Date: Jan. 22, 2015

(54) LIGANDS FOR RHODIUM CATALYZED REDUCTIVE CARBONYLATION OF ALCOHOLS

(71) Applicant: DOW GLOBAL TECHNOLOGIES LLC, Midland, MI (US)

(72) Inventors: Thomas P. Clark, Midland, MI (US); Heather A. Spinney, Midland, MI (US); Jason MacDonald, Bay City, MI (US); Clark H. Cummins, Midland, MI (US); Jerzy Klosin, Midland, MI (US); John R. Briggs, Midland, MI (US)

(73) Assignee: Dow Global Technologies LLC, Midland, MI (US)

(21) Appl. No.: 14/366,346

(22) PCT Filed: Dec. 20, 2012
(86) PCT No.: PCT/US2012/070970

§ 371 Date: Jun. 18, 2014

Related U.S. Application Data
(60) Provisional application No. 61/579,849, filed on Dec. 23, 2011.

(51) Int. Cl. B01J 31/22; C07C 29/09

(52) U.S. Cl. CPC B01J 31/2295; C07C 29/09; B01J 2231/4288
USPC 568/881; 502/162; 568/13; 549/220

(57) ABSTRACT

A catalytic system for reductive carbonylation of an alcohol that includes a rhodium complex, an iodide-containing catalyst promoter, and a supporting phosphorus-containing bidentate ligand for the rhodium complex containing at least one aromatic substituent covalently attached to at least one phosphorus of the supporting phosphorus-containing bidentate ligand in an ortho position with an alkoxy substituent or an aryloxy substituent.

This disclosure relates to the reductive carbonylation of alcohols, and in particular ligands for rhodium (Rh) catalyzed reductive carbonylation.

The reductive carbonylation of alcohols is illustrated by the conversion of methanol (MeOH) to acetaldehyde and 1,1-dimethoxyethane. The reaction is catalyzed by a Rh complex in the presence of 1,3-bis(diphenylphosphino)propane (dppp), which acts as a supporting ligand for the Rh complex (the combination of a supporting ligand and a rhodium complex will be referred to as the Rh catalyst), and methyl iodide (CH₃I) which acts as an iodide-containing catalyst promoter. The reaction occurs at 140° C. with a mixture of hydrogen (H₂) gas and carbon monoxide (CO) gas (e.g., synthesis gas (SynGas)) at a total pressure of 6.21 megapacal (MPa) (all pressures herein are gauge pressures). In the presence of an iodide-containing catalyst promoter of the present disclosure, the Rh catalyst converts the MeOH to acetaldehyde, 1,1-dimethoxyethane, and methyl acetate, where the molar selectivity of a combination of acetaldehyde and 1,1-dimethoxyethane is greater than 50%.

The iodide-containing catalyst promoter is preferably CH₃I, however it is known that other sources of iodide ions (F) are suitable for this reaction. For illustrative examples, see U.S. Pat. No. 4,727,200. The previously disclosed rhodium catalyst as described in U.S. Pat. No. 4,727,200 utilizes dppp as the supporting ligand and is selective towards acetaldehyde and 1,1-dimethoxyethane (“reductive carbonylation” products) over methyl acetate, but has an undesirably slow reaction rate. The present disclosure provides an improved reaction rate over the rhodium catalyst disclosed in U.S. Pat. No. 4,727,200. U.S. Pat. No. 4,843,145 discloses the use of bidentate ligands of phosphorus for palladium catalyzed ethylene/carbon monoxide copolymerization wherein at least one of the monovalent substituents of phosphorus is aromatic and is substituted in a position ortho to the phosphorus with a polar substituent.

Moloy and Wegman (Organometallics 1989, 8, 2883-2892) report a series of different supporting ligands, none of which are as effective as dppp. Gaemers and Sunley (WO 2004101487(A1)) disclose a series of rigid polydentate ligands which catalyze the reaction between methanol and SynGas to selectively form methyl acetate rather than reductive carbonylation products.

The present disclosure provides for the surprising discovery that specific polar substituents in the ortho position to the phosphorus provide an improvement in the rhodium-catalyzed reductive carbonylation process. The present disclosure provides for, among other things, a catalytic system for reductive carbonylation of an alcohol that includes a rhodium (Rh) complex; an iodide-containing catalyst promoter; and a supporting phosphorus-containing bidentate ligand for the rhodium catalyst containing at least one aromatic substituent covalently attached to at least one phosphorus of the supporting phosphorus-containing bidentate ligand, where the at least one aromatic substituent is substituted in an ortho position with an alkoxy substituent or an aryloxy substituent, and where the reductive carbonylation of the alcohol with CO gas and H₂ gas and the iodide-containing catalyst promoter by the system produces an acetal (a R₂C(OR′)₂ compound, where R′ is not H and thus a diether of a geminal diol); an aldehyde; an aldehyde and an acetal; or an aldehyde, an acetal and a homologous alcohol.

Specifically, the present disclosure describes the discovery of a catalytic system with improved activity and selectivity for homologation products, where the supporting phosphorus-containing bidentate ligand for the Rh complex is a compound of Formula I:

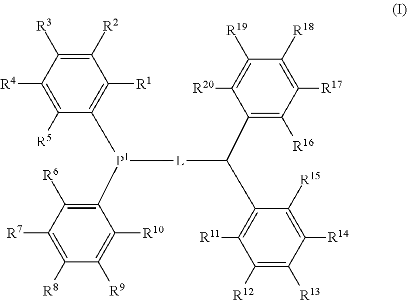

The phosphorus-containing bidentate supporting ligand includes an ortho-alkoxy or ortho-aryloxy substituent on at least one of the aryl groups (Ar), where at least one of R¹, R⁵, R⁶, R¹⁰, R¹¹, R¹⁵, R¹⁶ or R²⁰ is of the formula —OR²¹ where the oxygen (O) is covalently bonded to the Ar in the ortho position to the phosphorus and R²¹ is a hydrocarbyl group having C1 to C20, or a heterohydrocarbyl group having 1 to 20 atoms each independently selected from carbon (C) or a heteroatom, wherein each heteroatom is independently O, sulfur (S), silicon (Si), germanium (Ge), phosphorus (P) or nitrogen (N), and may themselves be substituted or unsubstituted as required by the valency of the heteroatom. It is to be understood that the supporting ligand may include additional P atoms, which may or may not be bound to a Rh or other metal atom.

Preferably, R²¹ should not be excessively bulky, such as isopropyl, since such ligand promoters do not generate catalysts that exhibit the highest reaction rates. As illustrated in the examples, below, the aryl group can contain one or more additional ring structures, either cyclic or polycyclic, each having 4 to 7 carbon atoms (C4 to C7) that is covalently bound to the aryl group at the meta-position to the respective P¹ and/or P² atom. For example, the R²¹ group can form a C4 to a C7 cyclic structure, including heterocyclic structures, by covalently bonding to the Ar in the adjacent meta-position to the P. Optionally, R²¹ can form a ring structure with the remaining hydrocarbyl or heterohydrocarbyl substituents.

Illustrative examples of ortho-alkoxy substituted Ar include:

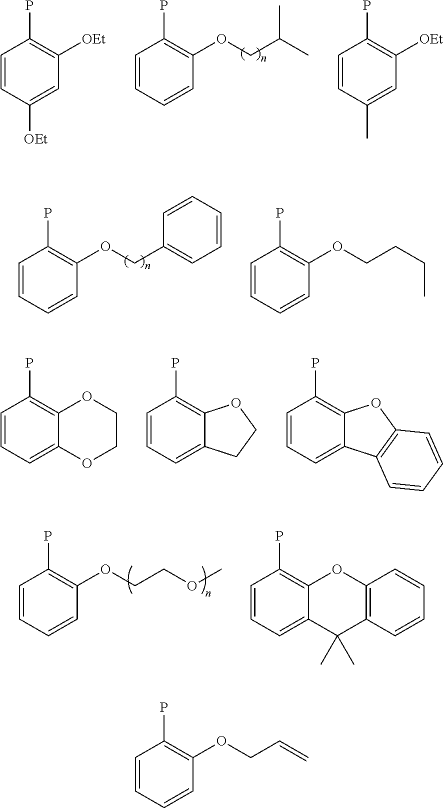

R², R³, R⁴, R⁷, R⁸, R⁹, R¹², R¹³, R¹⁴, R¹⁷, R¹⁸, and R¹⁹ are each independently a hydrogen (H), a hydrocarbyl group, an aromatic ring, a heteroaromatic ring or a halogen atom, or a heterocarbyl group selected from the group consisting of NR₂, OR and SR, where R is a hydrocarbyl group of C1 to C20, or heterohydrocarbyl group having 1 to 20 atoms each independently selected from C or a heteroatom, wherein each heteroatom is independently O, S, Si, Ge, P or N, and may themselves be substituted or unsubstituted as required by the valency of the heteroatom.

For Formula I, each aryl, heteroaryl, hydrocarbyl, heterohydrocarbyl, hydrocarbylene, and heterohydrocarbylene group independently is unsubstituted or substituted with one or more substituents R^{v}. Each R^{v} independently is a halogen atom, polyfluoroalkyl, unsubstituted C1 to C18 alkyl, F₃C—, FCH₂O—, F₂HCO—, F₃CO—, R₃Si, R₃Ge, RO, RS, RS(O), RS(O)₂, R₂P, R₂N, R₂C═N, NC, RC(O)O, ROC(O), RC(O)N(R), or R₂NC(O), or two of the R^{v} are taken together to form an unsubstituted C1 to C18 alkylene, wherein each R independently is an unsubstituted C1 to C18 alkyl. Optionally, two of the R^{v} are taken together to form a ring, where the ring can be cyclic or polycyclic.

The linking group, L, includes a chain linking the P¹ and P² atoms of 1 to 10 atoms optionally substituted with R^{v}. Preferably, the linking group, L, is selected from the group consisting of a hydrocarbylene group, a heterohydrocarbylene group and a ferrocenyl group. The hydrocarbylene has a chain linking the P¹ and P² atoms of 1 to 10 atoms which may be carbon (C1 to C10) or heteroatoms or combinations thereof linking the phosphorus (P) atoms. Up to 50 atoms can be covalently bonded to the hydrocarbylene. The up to 50 atoms includes C, O, S, Si, H, N, P and combinations thereof.

The heterohydrocarbylene has a chain of 1 to 10 atoms linking the P¹ and P² atoms. Each atom of the heterohydrocarbylene is independently a C or a heteroatom optionally substituted with R^{v}. Each heteroatom is independently selected from O, S, Si, Ge, P or N, wherein independently each heteroatom can be a substituted or unsubstituted (C1 to C18) hydrocarbyl or be part of a ring. Up to 50 atoms can be covalently bonded to the heterohydrocarbyl. The up to 50 atoms includes C, O, S, Si, H, N, chlorine (Cl), fluorine (F), bromine (Br), iodine (1) and combinations thereof.

The linker group, L, may also be part of more complex structures such as a ferrocenyl group. Optionally, two of the R^{v} used with the linking group, L, can be linked together to form a ring.

Illustrative examples of the —P¹-L-P²— moiety of Formula I include:

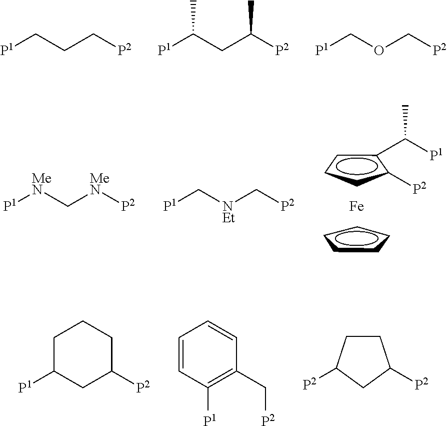

Specific examples of Formula I include:

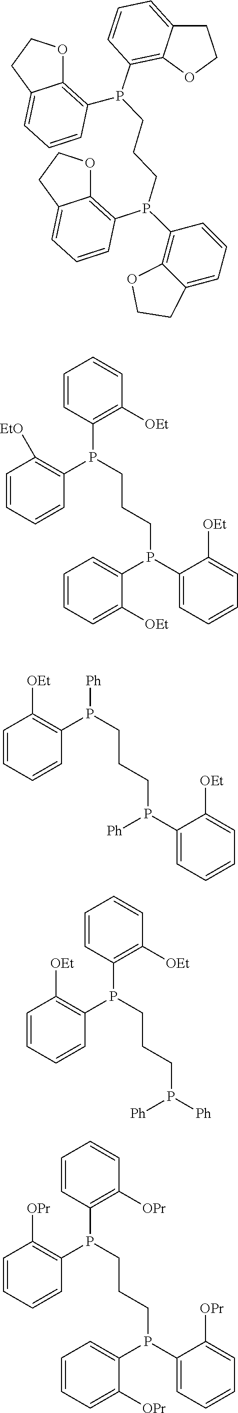

The alcohol, ROH, may be methanol (MeOH), ethanol (EtOH), or other primary alcohol, and is most preferably MeOH or EtOH. Reductive carbonylation includes reacting MeOH with H₂ gas and CO gas (e.g., a mixture of H₂ gas and CO gas such as SynGas) employing a Rh complex, methyl iodide (CH₃I) and the supporting ligand of Formula I to produce MeCHO, MeCH(OR)₂, EtOH or mixtures thereof, where R is a group derived from any alcohol present in the system, and most preferably is Me, Et, n-Pr and the like.

The Rh complex is a single Rh compound or a mixture of two or more Rh compounds. Examples include Rh metal, Rh salts and oxides, organo Rh compounds and coordination compounds of Rh. A preferred Rh complex of the present disclosure is (Acetylacetonato)dicarbonylrhodium(I) (Rh(acac)(CO)₂)).

An amount of Rh catalyst can vary for different applications. The Rh catalyst can be in a range of from 0.000001 mole percent (mol %) to 10.0 mol % relative to each mole of ROH, although an excess or deficiency of ligand may be employed relative to rhodium if desired. More preferably, the Rh catalyst can be in a range of from 0.001 mole percent (mol %) to 1.0 mol % relative to each mole of ROH. Most preferably, the Rh catalyst can be in a range of from 0.01 mole percent (mol %) to 0.10 mol % relative to each mole of ROH.

A Rh catalyst:iodide (I− ion) promoter mole ratio (moles of Rh catalyst:moles of the I− ion) is from 1:500 to 500:1, preferably from 1:300 to 300:1 and most preferably from 1:100 to 100:1. CH₃I is a preferred iodide-containing catalyst promoter.

A Rh catalyst:supporting ligand mole ratio is from 1:100 to 100:1, preferably from 10:1 to 1:10 and most preferably from 2:1 to 1:2.

Reaction conditions include a temperature of from 50° C. to 250° C., preferably from 100° C. to 170° C. and most preferably from 110° C. to 160° C., where 140° C. is most preferred.

Total reaction pressure, which includes the H₂ gas and the CO gas, is from 689.48 kilopascal (KPa, gauge) to 68.95 MPa, preferably from 1.72 MPa to 34.47 MPa and most preferably from 3.45 MPa to 17.24 MPa.

The H₂ gas to CO gas ratio (H₂:CO gas mixtures) is in a range from a 1:30 (vol:vol) ratio to a 30:1 (vol:vol) ratio. Preferably, the H₂ gas and CO gas ratio is in a range from a 1:8 ratio to an 8:1 ratio. Most preferably, the H₂ gas and CO gas ratio is in a range from a 3:1 ratio to a 6:1 ratio.

Reaction times vary depending upon the reaction parameters. The reaction can be a batch or continuous process reaction.

### EXAMPLES

All materials, unless noted otherwise, were obtained from Sigma-Aldrich®. Hydrogen (H₂) and carbon monoxide (CO) were obtained from Airgas. 1,3-Bis(dichlorophosphino)propane was obtained from Digital Specialty Chemicals. 2-Bromophenetole was obtained from Eastman. Toluene (TOL) was purified through a column of activated alumina followed by a column of Q5 copper oxide on alumina (Cu-0226 S, Englehard, BASF Corporation). All other solvents were anhydrous grade and were used without purification. Proton, carbon-13, and phosphorus-31 NMR spectra were obtained on one of four spectrometers: (1) Varian Mercury VXR-300, (2) Varian Mercury VX-400, (3) Varian MR-400, or (4) Varian VNMRS-500. Chemical shifts are in parts per million (ppm) relative to solvent peaks: ¹H═7.25 for CHCl₃ in CDCl₃ and 7.16 for C₆HD₅ in C₆D₆; ¹³C=77.2 for CDCl₃ and 128.4 for C₆D₆ Gas chromatography (GC) samples were run on a HP 6890 GC instrument with a J&W Scientific (Agilent Technologies) DB-1701 column (30 meter, 0.32 millimeter (mm) I.D.) using a flow of 1.0 milliliter/minute (mL/min) at a temperature of 35° C. for 2 min, followed by a temperature ramp of 20° C./min up to a maximum of 250° C.

### Synthesis of Supporting Phosphorus-Containing Bidentate Ligand

### Supporting Ligand Phosphorus-Containing Bidentate Example (SL Ex) 1

### 1,2-Bis(di-o-ethoxyphenylphosphino)ethane

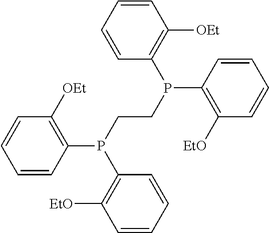 [SMILES: CCOc1ccccc1P(CCP(c1ccccc1OCC)c1ccccc1OCC)c1ccccc1OCC]

Prepare SL Ex1 as follows. Add ethoxybenzene (2.8 mL) to a glass jar with a PTFE-coated stirbar in a nitrogen (N₂) purged glovebox. To the contents of the jar add anhydrous tert-butylmethylether (MTBE). Add n-Butyllithium (BuLi) (8.0 mL, 2.5 molar (M) in hexanes) to the contents of the jar. Attach a reflux condenser to the jar and place the jar in a 60° C. aluminum heating block. Stir the contents of the jar for 8 hours (hr). Remove the condenser and cool the contents of the jar to −40° C. Add 1,2-bis(dichlorophosphino)ethane (0.71 mL, Strem) with hexane (10 mL) to a container and cool to −40° C. Add the contents of the container slowly to the contents of the jar. Warm the contents of the jar to 23° C. and stir for 72 hr.

Add the contents of the jar into degassed water (about 40 mL) and mix thoroughly. Separate etherate layer from aqueous layer and white solid. Rinse both layers twice with diethyl ether (Et₂O). Add methylene chloride (CH₂Cl₂, about 40 mL) to the aqueous layer to dissolve the solid. Separate the CH₂Cl₂ solution from the aqueous layer, dry over MgSO₄ and filter. Place under vacuum and collect SL Ex 1.

Analyze SL Ex 1 by NMR spectroscopy. ³¹P NMR spectroscopy shows a peak at −25.8 ppm, (referenced against H₃PO₄). ¹H and ¹³C NMR spectra confirm SL Ex 1 formation: ¹H NMR (500 MHz, C₆D₆) δ 1.18 (t, J=6.9 Hz, 12H), 2.28 (t, JH-P=4.0 Hz, 4H), 3.93 (m, 8H), 6.76-6.92 (m, 8H), 7.16-7.32 (m, 8H); ¹³C NMR (101 MHz, C₆D₆) δ 161.0 (C), 133.7 (CH), 129.7 (CH), 120.5 (CH), 111.1 (CH), 63.8 (OCH₂), 20.8 (PCH₂), 14.7 (CH₃); ³¹P NMR (202 MHz, C₆D₆) δ −25.8.

### SL, Ex 2

### 1,3-Bis(di-o-ethoxyphenylphosphino)propane

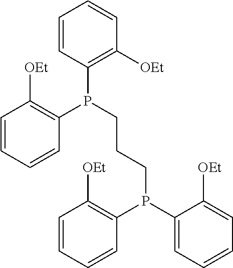 [SMILES: CCOc1ccccc1P(CCCP(c1ccccc1OCC)c1ccccc1OCC)c1ccccc1OCC]

Prepare SL Ex 2 as follows. Add 2-bromophenetole (8.0 g and 60 mL Et₂O) to a glass jar with a PTFE-coated stirbar in a N₂ purged glovebox. Cool the contents of the jar to −40° C. Add dropwise n-Butyllithium (BuLi) (17.5 mL of a 2.5 M solution in hexanes) to the contents of the jar. Stir the contents of the jar for 1 hr at 23° C. Filter the contents of the jar through a 20 micron polyethylene frit and rinsed 2× with hexanes to isolate lithiated ethoxybenzene (LiEt). Dry LiEt under vacuum for 1 hr at 30° C.

Add LiEt (3.17 g) to MTBE (40 mL) in a glass jar while stirring. Cool the contents of the glass jar to −40° C. Add 1,3-bis(dichlorophosphino)propane (1.45 g) and 10 mL hexanes to a glass container and cool to −40° C. Add the contents of the glass container slowly to the contents of the glass jar. Cool the contents of the glass jar about halfway through the addition. Stir the contents of the glass jar overnight at 23° C. in the N₂ purged glovebox. Slowly add degassed water (about 40 mL) to the contents of the jar and mix. Separate the etherate layer from the aqueous layer and SL Ex 2. Rinse suspension with MTBE. Add CH₂Cl₂ (15 mL) to the aqueous layer to dissolve SL Ex2. Separate the CH₂Cl₂ solution, dry over MgSO₄ and filter. Remove the solvent from the CH₂Cl₂ solution in vacuo.

Analyze SL Ex 2 by ¹H, ¹³C and ³¹P NMR spectroscopy to confirm formation. ¹H NMR (400 MHz, CDC₃) δ 7.10-7.20 (m, 8H), 6.70-6.90 (m, 811), 3.92 (m, 811), 2.30 (m, 4H), 1.64 (m, 2H), 1.20 (t, J=7.0 Hz, 12H); ¹³C{¹H} NMR (101 MHz, CDC₃) δ 161.0 (C), 133.5 (CH), 129.6 (CH), 126.8 (C), 120.6 (CH), 111.2 (CH), 63.9 (OCH₂), 27.0 (CH₂), 23.6 (CH₂), 14.8 (CH₃); ³¹P NMR (162 MHz, CDC₃) δ 32.5 ppm.

### SL Ex 3

### 3-(bis(2-ethoxyphenyl)phosphino)propyl)diphenyl-phosphine

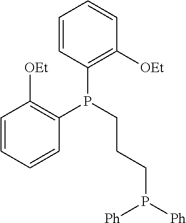 [SMILES: CCOc1ccccc1P(CCCP(c1ccccc1)c1ccccc1)c1ccccc1OCC]

Prepare SL Ex 3 as follows. Perform procedure in N₂-purged glovebox. Add LiEt (3.4 g) to MTBE (40 mL) in a glass jar while stirring with a PTFE-coated stir bar. Cool the contents of the glass jar to −40° C. Add PCl₃ (1.1 mL) to MTBE (20 mL) in a separate glass jar with stirring. Cool the contents of the second glass jar to −40° C. Add the contents of the second glass jar slowly to the contents of the first glass jar while maintaining the temperature below 0° C. Cool the contents of both jars about halfway through the addition. Stir the reaction mixture for 6 hr at 23° C. Filter mixture through 0.45 micron PTFE syringe frit to remove solids. Remove solvent from filtrate in vacuo to recover chlorophosphine, bis(2-ethoxyphenyl)chlorophosphine.

Dissolve bis(2-ethoxyphenyl)chlorophosphine in Et₂O (10 mL) in a glass jar while stirring with a PTFE-coated stir bar. Cool the contents of the glass jar to −40° C. Add LiAlH₄ (1.0 g) to Et₂O (40 mL) in a separate jar while stirring. Cool the contents of the second glass jar to −40° C. Add the contents of the first glass jar slowly to the contents of the second glass jar. Cool the contents of both jars about halfway through the addition. Stir the reaction mixture overnight at 23° C. Filter mixture through 0.45 micron PTFE syringe frit to remove solids. Quench reaction mixture by dropwise addition of 1 M 1HCl solution in water. Separate the Et₂O solution from the aqueous solution. Dry Et₂O solution over MgSO₄, filter and remove solvent in vacuo. Confirm formation of desired secondary phosphine, bis(2-ethoxyphenyl)phosphine by ³¹P NMR spectroscopy.

Dissolve bis(2-ethoxyphenyl)phosphine (1.9 g, 90% purity) in tetrahydrofuran (THF, 30 mL) in a glass jar containing a PTFE-coated stir bar. Cool the contents of the glass jar to −40° C. Add BuLi (2.8 mL of a 2.5 M solution in hexanes) dropwise to the contents to the glass jar. Stir for 1 hr at 23° C. Add 1,3-dichloropropane (10 mL) to THF (40 mL) in a separate glass jar containing a PTFE-coated stir bar. Add the contents the first glass jar slowly to the contents of the second glass jar over 30 min. Stir reaction mixture for 1.5 hr at 23° C. Quench reaction mixture with 2 mL of MeOH and remove solvent in vacuo overnight. Dissolve residue in Et₂O (30 mL) and add degassed water (30 mL). Separate the Et₂O solution from the aqueous solution. Dry Et₂O solution over MgSO₄, filter and remove solvent in vacuo. Characterize resultant oil by characterized by ¹H—, ³¹P—, and TOCSY1D-NMR spectroscopy. The oil is a mixture of the desired product ((2-ethoxyphenyl)₂PCH₂CH₂CH₂C1) and remaining 1,3-dichloropropane. Dry oil under vacuum for 4 hr at 40° C. and then use in next step of the reaction.

Add diphenylphosphine (0.88 mL, 5.1 mmol) to THF (30 mL) in a glass jar while stirring with a PTFE-coated stir bar. Cool the contents of the glass jar to −40° C. Add BuLi (2.1 mL of a 2.5 M solution in hexanes) slowly to the contents of the glass jar. Stir for 1 hour at 23° C. Dissolve oil from previous paragraph in THF (40 mL) in a separate glass jar containing a PTFE-coated stir bar. Add contents of first glass jar slowly to contents of the second glass jar over a period of 45 min. Stir reaction mixture for 2 hr at 23° C. Quench reaction mixture with 1 mL of MeOH and remove solvent in vacuo overnight. Suspend residue in Et₂O (40 mL) and add degassed water (20 mL). Stir vigorously for 5 min. Separate the Et₂O solution from the aqueous solution. Dry Et₂O solution over MgSO₄, filter and remove solvent in vacuo. Recrystallize resultant oil from CH₂Cl₂ hexanes (about 1:20) to provide SL Ex 3 as an off-white solid.

Analyze SL Ex 3 by ¹H, ¹³C and ³¹P NMR spectroscopy to confirm formation. ¹H NMR (400 MHz, CD₂Cl₂) δ 7.45-7.37 (m, 4H), 7.37-7.25 (m, 8H), 7.18 (m, 2H), 6.89 (t, J=7.5 Hz, 2H), 6.84 (m, 2H), 3.96 (m, 4H, OCH₂CH₃), 2.32 (t, J=7.7 Hz, 2H, PCH₂CH₂), 2.25 (t, J=7.6 Hz, 2H, PCH₂CH₂), 1.63 (m, 2H, PCH₂CH₂), 1.24 (t, J=6.9 Hz, 6H, OCH₂CH₃); ¹³C{¹H} NMR (100 MHz, CD₂Cl₂) δ 161.4 (d, J_{C-P}=11 Hz, 2C), 139.8 (d, J_{C-P})=14 Hz, 2C), 133.7 (d, J_{C-P}=10 Hz, 2CH), 133.2 (d, J_{C-P}=18 Hz, 2CH), 130.2 (s, 2CH), 128.95 (s, 2-4CH), 128.90 (s, 2CH), 127.0 (d, J_{C-P}=17 Hz, 2C), 121.0 (d, J_{C-P}=3 Hz, 2CH), 111.8 (s, 2CH), 64.4 (s, 2CH2), 30.2 (t, J_{C-P}=12 Hz, 2C, PCH₂CH₂), 27.03 (t, J_{C-P}=12 Hz, 2C, PCH₂CH₂), 23.6 (t, J_{C-P}=17 Hz, 2C, PCH₂CH₂); ³¹P {¹H} NMR (162 MHz, CD₂Cl₂) δ 17.3, 32.9 ppm.

### SL Ex 4

### 1,3-Bis(dihydrobenzofuranphosphino)propane

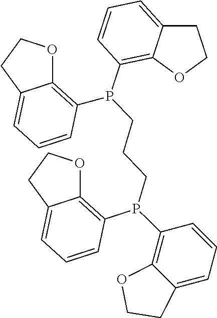 [SMILES: c1cc2c(c(P(CCCP(c3cccc4c3OCC4)c3cccc4c3OCC4)c3cccc4c3OCC4)c1)OCC2]

Prepare SL Ex 4 as follows. Add dihydrobenzofuran (2.7 g) to Et₂O (40 mL) in a glass jar with a PTFE-coated stirbar in a N₂ purged glovebox. Cool the contents of the glass jar to −40° C. Add BuLi (9.0 mL of a 2.5 M solution in hexanes) dropwise to the contents of the glass jar with stirring. Stir the contents of the glass jar for 72 hr at 23° C. Remove the solvent in vacuo. Isolate orange solid by rinsing with cold hexanes. Analyze the orange solid to confirm formation of aryllithium at a purity of 65%. Total solids=1.12 g. Dissolve the orange solid with Et₂O (40 mL) in a glass jar and cool to −40° C.

Add dropwise 1,3-bis(dichlorophosphino)propane (0.25 mL) to the contents of the glass jar. Stir the contents of the glass jar at 23° C. for 16 hr. Add 40 mL degassed water to the contents of the glass jar to quench the reaction. Vigorously stir the contents of the glass jar. Remove ether solution and rinse the water/solid mixture with ether. Extract SL Ex 3 into methylene chloride. Dry SL Ex 3 over MgSO₄, filter, and place under vacuum. Dry SL Ex 3 under vacuum for 3 hr to yield 0.60 g. Analyze SL Ex 3 by ¹H, ¹³C, APT, and ³¹P {¹H} NMR spectroscopy to confirm formation ¹H NMR (400 MHz, CD₂Cl₂) δ 7.16 (m, 4CH), 6.92 (m, 4CH), 6.76 (m, 4CH), 4.47 (t, J=8.7 Hz, 4CH₂CH₂O), 3.16 (t, J=8.7 Hz, 4CH₂CH₂O), 2.25 (m, 2CH₂P), 1.53 (m, CH₂CH₂P); ¹³C{¹H} NMR (100 MHz, CD₂Cl₂) δ 163.1 (d, J_{C-P}=13 Hz, 4C), 131.8 (d, J_{C-P}=9 Hz, 4CH), 127.2 (s, 4C), 125.8 (s, 4CH), 121.0 (d, J_{C-P}=3 Hz, 4CH), 118.0 (d, J_{C-P}=17 Hz, 4C), 71.6 (s, 4CH₂), 30.2 (s, 4CH₂), 27.1 (t, J_{C-P}=12 Hz, 2CH₂), 23.8 (s, CH₂); ³¹P {¹H} NMR (162 MHz, CD₂Cl₂) δ −36.8 ppm.

### SL Ex 5

### 1,3-Bis((o-ethoxyphenyl)phenylphosphino)propane

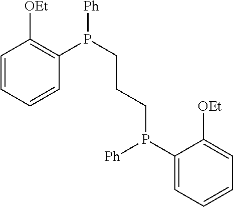 [SMILES: CCOc1ccccc1P(CCCP(c1ccccc1)c1ccccc1OCC)c1ccccc1]

Prepare SL Ex 5 as follows. Combine Ph(NEt₂)PCl1 (3.82 g) and THF (40 mL) in a first glass jar with a PTFE-coated stir bar in a N₂ purged glovebox. Cool the contents of the glass jar to −30° C. Add LiEt (2.27 g) to THF (30 mL) in a second glass jar while stirring with a PTFE-coated stir bar. Cool the contents of the second glass jar to −30° C. Add contents of second glass jar slowly to contents of first glass jar. Stir overnight at 23° C. Remove solvent in vacuo and triturate once with hexanes (40 mL). Dissolve resultant yellow oil in toluene (TOL, 60 mL) and filter through Celite® to remove solids. Cool filtrate to −30° C. Add HCl (18 mL of a 2 M solution in Et₂O) slowly to glass jar containing filtrate. Stir for 2 hr at 23° C. Filter solution through Celite® to remove solids. Remove solvents in vacuo and triturate with hexanes (40 mL). Dry resultant yellow oil for 1 hr under vacuum. Confirm formation of desired chlorophosphine, ClPPh(2-ethoxyphenyl) by ³¹P-NMR spectroscopy.

Add chlorophosphine, ClPPh(2-ethoxyphenyl) (3.73 g) to Et₂O (40 mL) in a glass jar containing a PTFE-coated stir bar. Cool the contents of the glass jar to −30° C. Add LiAlH₄ (0.535 g) slowly in small portions to contents of glass jar. Stir reaction mixture for 2 hr at 23° C. Filter through Celite® to remove excess LiAlH₄. Quench filtrate by slow addition of 1 M aqueous HCl solution (3 mL). Add degassed water (20 mL) and Et₂O (20 mL). Separate the Et₂O solution from the aqueous solution. Dry Et₂O solution over MgSO₄, filter and remove solvent in vacuo. Triturate resultant white solid with hexanes (30 mL) and farther dry under vacuum for 1 hr. Confirm formation of desired secondary phosphine, HPPh(2-ethoxyphenyl) by ³¹P-NMR spectroscopy.

Add secondary phosphine, HPPh(2-ethoxyphenyl), (2.45 g) to THF (40 mL) in a glass jar containing a PTFE-coated stir bar. Cool the contents of the glass jar to −30° C. Add BuLi (7.0 mL of a 1.6 M solution in hexanes) slowly to the contents of the glass jar while stirring. Stir for 2 hr at 23° C. Cool the contents of the glass jar again to −30° C. Add 1,3-dibromopropane (0.54 mL) dropwise to the contents of the glass jar. Stir for 1 hr at 23° C. Remove solvents in vacuo and take up resultant white residue in Et₂O (50 mL). Filter through Celite® to remove solids. Add degassed water (40 mL) to filtrate. Separate the Et₂O solution from the aqueous solution. Extract aqueous solution with additional Et₂O (30 mL), and combine the two Et₂O solutions. Dry Et₂O solution over MgSO₄, filter, and remove solvent in vacuo. Triturate resultant oily, white solid with hexanes (30 mL). Heat oily solid in MeOH (10 mL) for 2 hr at 60° C. Collect fine, white precipitate by filtration and dry under vacuum for 1 hr. Confirm by ³¹P-NMR spectroscopy that this is the desired material. Cool MeOH filtrate at −30° C. overnight. Collect precipitated solids by filtration and confirm by ³¹P-NMR spectroscopy that this is the desired material. Combine both crops of material for a total of 0.644 g. Note: SL Ex 5 exists as two diastereomers in solution, with varying ratios of each depending on the crop of material isolated.

Analyze SL Ex 5 by ¹H, ¹³C and ³¹P NMR spectroscopy to confirm formation. ¹H NMR (400 MHz, C₆D₆) δ 7.48-7.52 (m, 4H, PhH), 7.28-7.30 (m, 2H, ArH), 7.08-7.13 (m, 8H, ArH/PhH), 6.83 (t, J=7.2 Hz, 2H, ArH), 6.50 (dd, J=3.2 Hz & 8.0 Hz, 2H, ArH), 3.40-3.55 (m, 4H, OCH₂CH₃), 2.33-2.47 (m, 2H, PCH₂), 2.11-2.24 (m, 2H, PCH₂), 1.82 (septet, J=8.0 Hz, PCH₂CH₂), 0.94-0.98 (m, 6H, OCH₂CH₃); ¹³C{¹H} (101 MHz, C₆D₆) δ 161.42 (d, J_{PC}=11.4 Hz, C), 161.37 (d, J_{PC}=11.5 Hz, C), 140.28 (d, J_{PC}=14.9 Hz, C), 140.25 (d, J_{PC}=15.0 Hz, C), 133.83 (d, J_{PC}-19.9 Hz, CH), 133.74 (d, J_{PC}=19.7 Hz, CH), 133.41 (d, J_{PC}-8.8 Hz, CH), 133.24 (d, J_{PC}=8.2 Hz, CH), 130.28 (s, CH), 130.23 (s, CH), 128.72 (d, J_{PC}=10.0 Hz, CH), 128.70 (s, CH), 121.35 (d, J_{PC}=3.0 Hz, CH), 111.86 (s, CH), 64.06 (s, OCH₂CH₃), 29.11 (t, J_{PC}=12.8 Hz, PCH₂), 29.06 (t, J_{PC}=12.9 Hz, PCH₂), 23.82 (t, J_{PC}=18.3 Hz, PCH₂CH₂), 23.75 (t, J_{PC}=18.4 Hz, PCH₂CH₂), 14.98 (s, OCH₂CH₃); ³¹P{¹H} NMR (162 MHz, C₆D₆) δ −24.7 (33.3%), −24.9 (66.6%) ppm.

### SL Ex 6

### 1,3-bis(bis(2-methyl-2,3-dihydrobenzofuran-7-yl)phosphino)propane

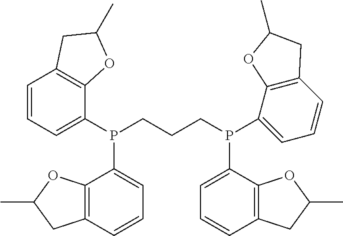

Prepare SL Ex 6 as follows: Dissolve 2,3-dihydro-2-methylbenzofuran (5.0 mL (39 mmol) from TCI) in MTBE (50 mL) and cool in a freezer at −40° C. in a N₂ purged glovebox. Remove the solution from the freezer and add n-butyllithium (15 mL of a 2.5 M solution in hexanes) dropwise to the stirring solution. Attach a reflux condenser and stir the solution overnight at 55° C. Remove solvent in vacuo. Use the intermediate without further purification. Analyze the intermediate by ¹H NMR spectroscopy in d₈-THF to estimate the quantity of aryllithium present. Use this value to estimate the amount of chlorophosphine to add. Dissolve the crude aryllithium in diethyl ether (40 mL). Place the solution in the freezer at −40° C. for 30 minutes. Remove from the freezer and add 1.1 mL (6.7 mmol) of 1,3-bis(dichlorophosphino)propane dropwise. Stir the resultant mixture for 5 hours at room temperature. Quench the reaction mixture by adding 30 mL of degassed water. Discard the ether solution and dissolve the solid in methylene chloride (40 mL). Isolate this organic layer and dry over MgSO₄. Filter the mixture and remove the solvent in vacuo. Analyze the resultant solid (0.91 g, 1.4 mmol, 21%) by NMR spectroscopy in CD₂Cl₂ to confirm the structure. Several peaks are present in the ³¹P{¹H} NMR spectrum between −36.6 and −35.5 ppm. This is consistent with the formation of diastereomers of the ligand from the use of a racemic 2-methyl-2,3-dihydrobenzofuran.

### SL Ex 7

### 1,3-bis(bis(dibenzo[b,d]furan-4-yl)phosphino)propane

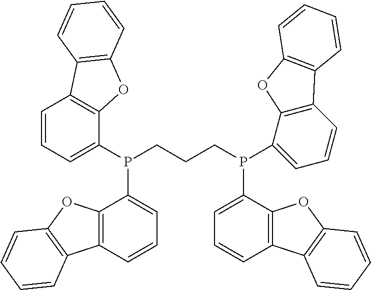 [SMILES: c1ccc2c(c1)oc1c(P(CCCP(c3cccc4c3oc3ccccc34)c3cccc4c3oc3ccccc34)c3cccc4c3oc3ccccc34)cccc12]

Prepare SL Ex 7 as follows: Dissolve dibenzofuran (2.5 g, 15 mmol) in diethyl ether (40 mL) and cool in a freezer at −40° C. in a N₂ purged glovebox. Remove the solution from the freezer and add n-butyllithium (5.9 mL of a 2.5 M solution in hexanes) dropwise to the stirring solution. Stir the resultant solution overnight at room temperature. Isolate the solid by filtration and drying under vacuum. Suspend the solid (1.6 g, about 7.5 mmol) in diethyl ether (40 mL) and cool in a freezer at −40° C. Remove the suspension from the freezer and add 1,3-bis(dichlorophosphino)propane (0.31 mL, 1.86 mmol) dropwise. Stir the resultant mixture overnight. Quench the reaction mixture by adding 20 mL of degassed water. Dry the organic layer over MgSO₄, filter, and remove solvent in vacuo. Suspend the solid in methanol and heat to 50° C. Filter the resulting white suspension and rinse with methanol. Dry the white solid overnight under vacuum. Analyze the product (1.12 g, 1.45 mmol, 78% yield) by NMR spectroscopy in CD₂Cl₂ to confirm identity. Desired product with a trace of dibenzofuran present. ¹H NMR (400 MHz, CD₂Cl₂) δ 7.95 (d, J_{H-H}=7.5 Hz, 4H, ArH), 7.89 (d, J_{H-H}=7.9 Hz, 4H, ArH), 7.49 (m, 4H, ArH), 7.45-7.30 (m, 12H, ArH), 7.19 (t, J_{H-H}=7.5 Hz, 4H, ArH), 2.84 (m, 4H, 2PCH₂), 1.91 (m, 2H, PCH₂CH₂); ¹³C {¹H} NMR (100 MHz, CD₂Cl₂) δ 158.8 (d, J_{C-P}=12 Hz, 4C-O), 156.5 (s, 4C-O), 131.7 (d, J_{C-P}=11 Hz, 4CH), 127.8 (s, 4CH), 124.5 (s, 4C), 124.3 (s, 4C), 123.5 (d, J_{C-P}=4 Hz, 4CH), 123.4 (s, 4CH), 121.9 (s, 4CH), 121.2 (s, 4CH), 120.8 (d, J_{C-P}=20 Hz, 4C-P), 112.3 (s, CH), 27.3 (t, J_{C-P}=12 Hz, 2PCH₂), 24.0 (t, J_{C-P}=18 Hz, PCH₂CH₂); ³¹P {¹H} NMR (162 MHz, CD₂Cl₂) δ −36.5 ppm.

### SL Ex 8

### 13-bis(di-o-propoxyphenylphosphino)propane

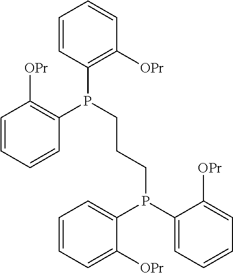

Prepare SL Ex 8 as follows. Combine 2-bromophenol (10.0 g), 1-bromopropane (5.25 mL), and acetonitrile (40 mL) in a 100-mL round bottom flask. Add potassium carbonate (24.0 g) and stir for 5 minutes. Attach reflux condenser to flask and heat mixture at 85° C. overnight. Cool mixture and filter through a medium porosity glass fit to remove solids. Remove acetonitrile from filtrate in vacuo and take up resultant yellow oil in CH₂Cl₂. Combine solution with aqueous 1.0 M NaOH (30 mL) and separate the two layers. Wash aqueous layer with 2×20 mL portions of CH₂Cl₂. Combine organic layers and dry over MgSO₄. Filter through medium porosity glass fit to remove solids. Remove solvent from filtrate in vacuo to recover 1-bromo-2-(n-propoxy)benzene.

Combine 1-bromo-2-(n-propoxy)benzene (10.6 g) and Et₂O (50 mL) in a glass jar containing a PTFE-coated stir bar in a N₂ purged glovebox. Cool the contents of the jar in the glove box freezer (−10° C.) for 1 hour. Remove jar from freezer and add n-butyllithium (34 mL of a 1.6 M solution in hexanes) slowly to the cold solution. Stir the resultant solution overnight at room temperature. Remove solvent in vacuo and suspend the resultant yellow solid in hexanes (40 mL). Collect solid by filtration and wash with an additional 40 mL of hexanes. Dry solid under vacuum for 2 hours and confirm formation of 1-lithio-2-(n-propoxy)benzene by ¹H NMR spectroscopy.

Combine 1-lithio-2-(n-propoxy)benzene (6.69 g) and THF (50 mL) in a glass jar containing a PTFE-coated stir bar. Cool the contents of the jar in the glove box freezer (−10° C.) for 1 hour. Remove jar from freezer and add dimethyl phosphoramidous dichloride ((NMe₂)PCl₂, 2.7 mL) dropwise to the cold solution. Stir the resultant solution overnight at room temperature. Remove solvent in vacuo and triturate with 30 mL of hexanes. Take resultant yellow oil up in toluene (40 mL) and filter through Celite® to remove LiCl. Remove solvent from filtrate in vacuo and triturate with 40 mL of hexanes. Confirm formation of (NMe₂)P(o-propoxyphenyl)₂ (yellow oil) by ³¹P NMR spectroscopy.

Combine (NMe₂)P(o-propoxyphenyl)₂(7.38 g) and toluene (50 mL) in a glass jar containing a PTFE-coated stir bar. Cool the contents of the jar in the glove box freezer (−10° C.) for 1 hour. Remove jar from freezer and add HCl (2.0 M solution in Et₂O, 21 mL) slowly to the solution while stirring. Stir the resultant solution for 3 hours at room temperature. Filter through Celite® to remove ammonium salts. Remove solvent from filtrate in vacuo and triturate with 40 mL of hexanes. Confirm formation of ClP(o-propoxyphenyl)₂ (yellow oil) by ³¹P NMR spectroscopy.

Combine ClP(o-propoxyphenyl)₂ (5.84 g) and Et₂O (40 mL) in a glass jar containing a PTFE-coated stir bar. Cool the contents of the jar in the glove box freezer (−10° C.) for 1 hour. Remove jar from freezer and add LiAlH₄ (2.0 g) as a solid to the solution in small portions. Stir the resultant mixture for 3 hours at room temperature. Filter through Celite® to remove unreacted LiAlH₄. Quench the filtrate with 2 mL of 1.0 M solution of aqueous HCl followed by 20 mL of degassed water. Add additional Et₂O (20 mL) and separate organic and aqueous layers. Extract aqueous layer with an additional 30 mL portion of Et₂O. Combine Et₂O fractions and dry over MgSO₄. Filter to remove solids. Remove solvent from filtrate in vacuo and triturate with 40 mL of hexanes. Confirm formation of HP(o-propoxyphenyl)₂ (white solid) by ³¹P NMR spectroscopy.

Combine HP(o-propoxyphenyl)₂ (1.88 g) and THF (40 mL) in a glass jar containing a PTFE-coated stir bar. Cool the contents of the jar in the glove box freezer (−10° C.) for 1 hour. Remove jar from freezer and add n-butyllithium (4.3 mL of a 1.6 M solution in hexanes) slowly to the cold solution. Stir resultant red solution for 2 hours at room temperature. Add 1,3-dibromopropane (0.32 mL) dropwise with stirring to the solution. Stir resultant yellow solution for 2 hours at room temperature. Remove solvent in vacuo and triturate twice with 30 mL of hexanes. Take resultant yellow oil up in toluene (40 mL) and filter through Celite® to remove LiBr. Rinse solids collected in filter with additional toluene (10 mL). Remove solvent from filtrate in vacuo and triturate with 30 mL of hexanes. Suspend resultant white solid in methanol and heat at 60° C. for 2 hours. Collect SL Ex 8 by filtration. Place methanol filtrate in freezer (−10° C.) overnight. Collect crystals of SL Ex 8 by filtration. Concentrate methanol filtrate under vacuum and return solution to freezer (−10° C.) for 16 hours. Collect second crop of crystals of SL Ex 8 by filtration. Total yield of product=0.685 g.

Analyze SL Ex 8 by ¹H, ¹³C, and ³¹P NMR spectroscopy to confirm formation. ¹H NMR (500 MHz, C₆D₆) δ 7.41 (m, 4H, ArH), 7.13 (m, 4H, ArH), 6.82 (t, J_{HH}=9.0 Hz, 4H, ArH), 6.56 (dd, J_{HH}=10.0 Hz, 4.0 Hz, 4H, ArH), 3.50 (m, 8H, OCH₂), 2.52 (broad t, J=10.0 Hz, 4H, PCH₂), 1.97 (m, 2H, PCH₂CH₂), 1.48 (m, 8H, OCH₂CH₂), 0.79 (t, J_{HH}=9.5 Hz, 12H, CH₃). ¹³C{¹H} NMR (126 MHz, C₆D₆) δ 161.8 (d, J_{PC}=12.6 Hz, Ar), 134.0 (d, J_{PC}=10.0 Hz, ArH), 129.8 (s, ArH), 128.1 (hidden under C₆D₆, Ar), 121.2 (d, J_{PC}=3.8 Hz, ArH), 111.7 (s, ArH), 70.1 (s, OCH₂), 27.9 (dd, J_{PC}=13.8 Hz, 12.6 Hz, PCH₂), 24.6 (t, J_{PC}=18.9 Hz, PCH₂CH₂), 23.2 (s, OCH₂CH₂) 11.2 (s, CH₃); ³¹P NMR (162 MHz, C₆D₆) δ 32.5 (s) ppm.

### SL Comparative Example A (ComEx A)

### 1,3-Bis(bis(p-ethoxyphenyl)phosphino)propane

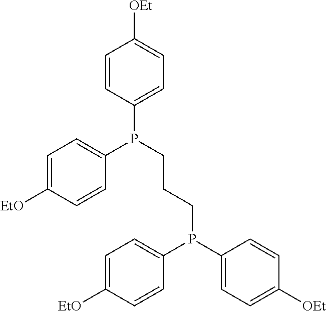 [SMILES: CCOc1ccc(P(CCCP(c2ccc(OCC)cc2)c2ccc(OCC)cc2)c2ccc(OCC)cc2)cc1]

Prepare SL ComEx A as follows. Combine 1-bromo-4-ethoxybenzene (1.33 mL) and Et₂O (30 mL) in a glass jar with a PTFE-coated stir bar in a N₂ purged glovebox. Add magnesium turnings (0.256 g) and 10 drops of 1,2-dibromoethane to contents of glass jar. Stir vigorously for 2.5 hr at 23° C. Filter through Celite® to remove solids, transfer filtrate to glass jar, and cool contents of jar to −30° C. Add 1,3-bis(dichlorophosphino)propane (0.35 mL) to Et₂O (10 mL) in a separate glass vial. Cool contents of vial to −30° C. Add contents of glass vial slowly to contents of glass jar. Add THF (30 mL) to glass jar to bring more material into solution. Stir overnight at 23° C. Filter through Celite® to remove solids. Remove solvent from filtrate in vacuo and take up resultant yellow residue in Et₂O (50 mL). Add degassed water (30 mL) to Et₂O solution. Separate Et₂O solution from aqueous solution. Extract aqueous solution with additional Et₂O (30 mL), and combine the two Et₂O solutions. Dry Et₂O solution over MgSO₄, filter, and remove solvent in vacuo. Triturate resultant yellow oil with hexanes (30 mL). Dissolve oil in hot MeOH (10 mL, 65° C.) and store MeOH solution at −30° C. overnight. Decant MeOH from precipitated solids. Triturate yellow solids with hexanes (30 mL) and further dry material for 1 hr under vacuum. Obtain 0.368 g of sticky, yellow solid (pure compound by ¹H, ¹³C and ³¹P NMR spectroscopy).

Analyze SL ComEx A by ¹H, ¹³C and ³¹P NMR spectroscopy to confirm formation. ¹H NMR (400 MHz, C₆D₆) δ 7.40-7.44 (m, 8H, ArH), 6.79 (d, J_{HH}=8.4 Hz, 8H, ArH). 3.59 (q, J_{HH}=7.2 Hz, 8H, OCH₂CH₃), 2.18 (t, J=7.6 Hz, 4H, P—CH₂), 1.74-1.88 (m, 2H, P—CH₂CH₂), 1.10 (t, J_{HH}=6.8 Hz, 12H, OCH₂CH₃); ¹³C{¹H} NMR (101 MHz, C₆D₆) δ 160.3 (s, Ar), 134.9 (d, J_{PC}20.2 Hz, ArH), 131.0 (d, J_{PC}=12.3 Hz, Ar), 115.4 (d, J_{PC}=7.3 Hz, ArH), 63.6 (s, OCH₂CH₃), 31.2 (t, J_{PC}=12.3 Hz, PGH₂), 23.5 (t, J_{PC}=17.5 Hz, PCH₂CH₂), 15.2 (s, OCH₂CH₃); ³¹P {¹H} NMR (162 MHz, C₆D₆) δ 20.6 ppm.

### SL ComEx B

### 1,8-Bis(diphenylphosphino)naphthalene

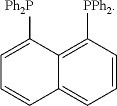

SL ComEx B is synthesized following a procedure similar to that reported in Synth. Comm. 1995, 25, 1741-1744. Prepare SL ComEx B (1,8-Bis(diphenylphosphino)naphthalene) as follows. Add BuLi (6.20 mL of a 2.5 M solution in hexanes) to a glass jar with a PTFE-coated stirbar in a N₂ purged glovebox. Add diethylether (10 mL) to the contents of the glass jar and cool to −40° C. Slowly add 1-bromonaphthalene (1.80 mL) to the contents of the glass jar. Stir the contents of the glass jar for approximately 15 min until the contents of the glass jar reaches 23° C. Cool the contents of the glass jar to −40° C. Separate the yellow solution and the solid in the glass jar by syringe. Add hexanes (15 mL) to the contents of the glass jar to rinse the solid. Cool the contents of the glass jar and remove by syringe. Repeat the rinse process. Dry the contents of the glass jar under vacuum to produce a white solid. Store the white solid in a N₂ purged glovebox.

Add 10 mL hexanes to the white solid in a glass jar. Slowly add BuLi (6.0 mL, 2.5 M in hexanes) to the contents of the glass jar. Slowly add tetramethylethylenediamine (2.3 mL) to the contents of the glass jar and stir to produce an orange solution. Attach a reflux condenser to the jar and place jar in a 77° C. aluminum heating block. Stir the contents of the glass jar for 3 hr. Cool the contents of the glass jar to 25° C. Add THF (15 mL) to the contents of the glass jar. Place the glass jar in a −40° C. freezer for 15 min. Add dropwise chlorodiphenylphosphine (5.9 mL in 10 mL THF) to the contents of the glass jar over 1 hr. Stir the contents of the glass jar for 1 hr at 23° C.

Slowly add the contents of the glass jar to distilled water. Isolate the organic layer from the glass jar and extract the aqueous layer from the glass jar with methylene chloride (20 mL). Combine the organic layer and the methylene chloride and dry over MgSO₄. Filter and remove solvent in vacuo for 18 hr to produce a solid. Dissolve the solid in 25 mL of hot benzene (60° C.) and add about 30 mL of MeOH to make a mixture. Cool the mixture and filter to isolate Ex 4. Wash Ex 4 with Cold MeOH.

Analyze SL ComEx B by ¹H, ¹³C, and ³¹P NMR spectroscopy: ¹H NMR (500 MHz, CDCl₃) δ 7.2-7.5 (m, 23H), 7.91 (m, 2H); ³¹P NMR (202 MHz, CDCl₃) δ −13.5.

### Methanol Reductive Carbonylation (MRC)

### MRC Ex 6 with SL Ex 1

Add (Acetylacetonato)dicarbonylrhodium(I) (25 milligram (mg), 0.01 mol. %, Rh(acac)(CO)₂) and SL Ex 1 (56 mg) to a glass vial in a N₂ purged glovebox. Add TOL (TOL, 4.1 mL) to the contents of the glass vial and mix. Add MeOH (15 mL) to the contents of the glass vial mix thoroughly to dissolve the Rh(acac)(CO)₂ and SL Ex 1 (solution 1). Add MeOH (25 mL) to CH₃I (0.30 mL, 0.5 mol. %) to a second vial (solution 2). Take solution 1 up in a first syringe and take solution 2 up in a second syringe. Inject solutions 1 and 2 into a Hastelloy C Parr reactor (“reactor,” with a Hastelloy C bottom valve to drain contents) open to air through a 3.35 mm valve. Close the valve and pressurize the reactor to 689.48 KPa N₂ and vent to remove oxygen; repeat.

While stirring at 600 rpm, pressurize the reactor to 2.07 MPa H₂ with a Brooks mass flow controller controlled by a Camile 2000. Heat the reactor to 140° C. Once the temperature of the contents of the reactor reaches 135° C., increase reactor pressure to 6.21 MPa with SynGas 1:1 (vol H₂:vol CO). Maintain the pressure at 6.21 MPa with the SynGas (1:1). After 2 hr, stop the reaction by closing the mix of SynGas (1:1) and turning the reactor temperature to below 40° C. Vent the reactor.

Measure the product distribution by GC in dioxane with an FID detector: column temp=35° C. for 2 min, 20° C./min to 250° C.; flow=1.0 mL/min, column=DB-1701, 30 m, 0.32 mm I.D. Assign the GC peaks by comparison with authentic samples. Determine yields by comparison with TOL as an internal standard: reductive carbonylation products 1,1-Dimethoxyethane=5.26%, acetaldehyde=0.39%, MeOH=83.6%, Methyl acetate=4.94%, average reductive carbonylation turnover frequency (ARCTF) (h⁻¹)=283. Mass balance (excluding dimethylether)=94.2%. Total CO added to the reactor: 2.53 g. Total H₂ added to the reactor: 0.39 g.

### MRC Ex 7 with SL Ex 2

Repeat MRC Ex 6, but with changes: use 50 mg of Rh(acac)(CO)₂ (0.02 mol. %); use 114 mg of SL Ex 2 (0.02 mol. %); of the 40 mL of anhydrous MeOH add about 17 mL to the vial and mix; add N₂ to pressurize the reactor to 1.38 MPa and vent. The CO flow was monitored at different times: at 30 min, the CO flow rate into the reactor was 0.018 g/min; at 48 min the rate was 0.022 g/min; at 90 min the rate was 0.033 g/min; and at 120 min, the rate was 0.030 g/min.

Measure the product distribution by GC in dioxane as in MRC Ex 6. Determine yields using TOL as an internal standard: reductive carbonylation products 1,1-Dimethoxyethane 8.81%, acetaldehyde=2.82%, MeOH=62.2%, methyl acetate=2.82%, ARCTF (h⁻¹)=291, mass balance=97.1%. Total CO added to the reactor: 4.42 g. Total H₂ added to the reactor: 0.49 g.

### MRC Ex 8 with SL Ex 2

Repeat MRC Ex 6, but with changes: use 50 mg of Rh(acac)(CO)₂ (0.02 mol. %); use 114 mg of SL Ex 2 (0.02 mol. %); of the 40 mL of anhydrous MeOH add about 17 mL to the vial and mix; add N₂ to pressurize the reactor to 1.38 MPa and vent. While stirring at 600 rpm, pressurize the reactor to 2.76 MPa H₂ as described in MRC Ex 6. Heat the reactor to 140° C. Once the temperature of the contents of the reactor reaches 135° C., increase reactor pressure to 6.21 MPa with SynGas 1:1 (vol H₂:vol CO). Maintain the pressure at 6.21 MPa with the SynGas (0.92:1). The CO flow is monitored during the reaction: at 20 min, the CO flow rate into the reactor is 0.042 g/min; at 40 min, the rate is 0.049 g/min; at 60 min, the rate is 0.042 g/min. After 1 hr stop the reaction by closing the mix of SynGas (0.92:1) and turning the reactor temperature to below 40° C. Vent the reactor.

Measure the product distribution by GC in dioxane as in MRC Ex 6. Determine yields using TOL as an internal standard: reductive carbonylation products 1,1-Dimethoxyethane=9.19%, acetaldehyde=1.58%, MeOH=60%, methyl acetate=1.29%, ARCTF (h⁻¹)=538, mass balance=92%. Total CO added to reactor=3.85 g; total H₂ added to reactor=0.50 g.

### MRC Ex 9 with SL Ex 3

Repeat MRC Ex 6, but with changes: use 50 mg of Rh(acac)(CO)₂ (0.02 mol. %); use 100 mg of SL Ex 3 (0.02 mol. %); of the 40 mL of anhydrous MeOH add about 17 mL to the vial and mix; add N₂ to pressurize the reactor to 1.38 MPa and vent. While stirring at 600 rpm, pressurize the reactor to 2.07 MPa H₂ as described in MRC Ex 6. Repeat the remaining steps of MRC Ex 6.

Measure the product distribution by GC in dioxane as in MRC Ex 6. Determine yields using TOL as an internal standard: reductive carbonylation products 1,1-Dimethoxyethane=9.08, acetaldehyde=2.13%, MeOH=56.4%, methyl acetate=0.94%, ARCTF (h⁻¹)=285, mass balance=88%. Total CO added to reactor=4.32 g; total H₂ added to reactor=0.49 g.

### MRC Ex 10 with SL Ex 4

Repeat MRC Ex 6, but with changes: use 25 mg of Rh(acac)(CO)₂ (0.01 mol. %); use 60 mg of SL Ex 4 (0.01 mol. %); of the 40 mL of anhydrous MeOH add about 18 mL to the vial and mix; SL Ex 4 did not go fully into solution and some of the solid remained behind when the mixture was taken up into the syringe; add N₂ to pressurize the reactor to 1.38 MPa and vent; while stirring at 600 rpm, add H₂ to increase reactor pressure to 2.76 MPa; reaching about 2.41 MPa raise the temperature of the reactor to 140° C.; after the temperature reached over 125° C., SynGas 1:1 (vol H₂:vol CO) was added to increase the reactor pressure to 6.21 MPa. Total amounts added in this step: 1.305 g CO and 0.095 g H₂; the contents of the reactor were stirred for 1 hr at 140° C.; the CO feed rate was approximately 0.042 g/min after 20 min, 0.032 g/min after 30 min, 0.023 g/min after 45 min, and 0.020 g/min after 60 min. After 1 hr, the reaction was stopped by stopping the SynGas and turning the reactor temperature to below 50° C.

Measure the product distribution by GC in dioxane as in MRC Ex 6. Determine yields using TOL as an internal standard: reductive carbonylation products 1,1-Dimethoxyethane=7.57%, acetaldehyde 1.07%, ethanol=0.06%, MeOH=67.8%, methyl acetate=0.37%, ARCTF (h⁻¹)=870, mass balance=92.3%. Total CO added to reactor=3.22 g, total H₂ added to reactor=0.46 g.

### MRC Ex 11 with SL Ex 4 at Different Level of CH₃I

Repeat MRC Ex 10, but with changes: use 0.05 mL CH₃I (0.17 mol % relative to methanol). The CO feed rate is approximately 0.016 g/min after 20 min, 0.012 g/min after 40 min, and 0.010 g/min after 60 min.

Measure the product distribution by GC in dioxane as in MRC Ex 6. Determine yields using TOL as an internal standard: reductive carbonylation products 1,1-Dimethoxyethane=3.62%, acetaldehyde=0.20%, ethanol=0.02%, MeOH=85%, methyl acetate=0.13%, ARCTF (h⁻¹)=384, mass balance=96.6%. Total CO added to reactor=1.906 g; total H₂ added to reactor=0.374 g.

### MRC Example 12

### SL Ex 2 at Different Level of CH₃I

Repeat MRC Ex 6, but with changes: use 60 mg of SL Ex 2; of the 40 mL of anhydrous MeOH add about 18 mL to the vial and mix; use 0.9 mL of CH₃I with remaining MeOH.

While stirring at 600 rpm, pressurize the reactor to 2.76 MPa H₂ (total H₂ added 0.237 g). At 2.14 MPa raise the temperature of the reactor to 140° C. After the temperature reaches 125° C., increase the pressure of the reactor to 6.21 MPa by adding SynGas 1:1 (vol. H₂:Vol. CO). Maintain the pressure at 6.21 MPa by confeeding a mix of SynGas 0.93:1. Stir the reaction for 1 hour at 140° C. The CO feed rate was 0.010 g/min after 30 minutes. The rate appeared to slowly get slower over time (down to about 0.006 g min after 1 hour). After 1 hour, stop the reaction by closing the gas feeds and turning the reactor temperature to below 40° C. Vent the reaction once the temperature drops below 40° C.

Measure the product distribution by GC in dioxane as in MRC Ex 6. Determine yields using TOL as an internal standard: reductive carbonylation products 1,1-dimethoxyethane=9.32%, acetaldehyde=2.77%, MeOH=54.7%, methyl acetate=0.76%, ARCTF (h⁻¹)=1209, mass balance=87.0%. Total CO added to reactor=4.169 g, total H₂ added to reactor=0.523 g.

### MRC Ex 13 with SL Ex 5

Repeat MRC Ex 6, but with changes: use 52 mg of Rh(acac)(CO)₂ (0.02 mol. %); use 100 mg of SL Ex 5 (0.02 mol. %); of the 40 mL of anhydrous MeOH add about 17 mL to the vial and mix; add N₂ to pressurize the reactor to 1.38 MPa and vent. While stirring at 600 rpm, pressurize the reactor to 2.07 MPa H₂ as described in MRC Ex 6. Heat the reactor to 140° C. Once the temperature of the contents of the reactor reaches 135° C., increase reactor pressure to 6.21 MPa with SynGas 1:1 (vol. H₂:vol CO). Maintain the pressure at 6.21 MPa with the SynGas (1:1). After 2 hr stop the reaction by closing the mix of SynGas (1:1) and turning the reactor temperature to below 40° C. Vent the reactor.

Measure the product distribution by GC in dioxane as in MRC Ex 6. Determine yields using TOL as an internal standard: reductive carbonylation products 1,1-Dimethoxyethane=6.43%, acetaldehyde=1.36%, MeOH=59.2%, methyl acetate=1.49%, ARCTF (h⁻¹)=195, mass balance=83%. Total CO added to reactor=3.61 g; total H₂ added to reactor=0.44 g.

### MRC Ex 14 with SL Ex 6

Repeat MRC Ex 6, but with changes: use 50 mg of Rh(acac)(CO)₂; use SL Ex 6 (130 mg). Of the 40 mL of anhydrous MeOH add about 18 mL to the vial and mix (solution 1) and use the remaining MeOH with the CH₃I (0.30 mL, 0.5 mol. %) to form solution 2.

While stirring at 600 rpm, pressurize the reactor to 2.76 MPa H₂ (total H₂ added=0.232 g). At 2.14 MPa raise the temperature of the reactor to 140° C. After the temperature reaches 125° C., increase the pressure of the reactor to 6.21 MPa by adding SynGas 1:1 (vol. H₂:Vol. CO) Maintain the pressure at 6.21 MPa by confeeding a mix of SynGas 1:1 for 20 minutes and 0.91:1 for the next 40 minutes. Stir the reaction for 1 hour at 140° C. The CO feed rate was 0.047 g/min after 20 minutes, 0.037 g/min after 40 minutes, and 0.032 g/min after 60 minutes. After 1 hour, stop the reaction by closing the gas feeds and turning the reactor temperature to below 50° C. Vent the reaction once the temperature drops below 50° C.

Measure the product distribution by GC in dioxane as in MRC Ex 6. Determine yields using TOL as an internal standard: reductive carbonylation products 1,1-Dimethoxyethane=8.71%, acetaldehyde 1.41%, ethanol=0.09%, MeOH=64.9%, methyl acetate 0.89%, ARCTF (h⁻¹)=511, mass balance 94.3%. Total CO added to reactor=3.64 g, total H₂ added to reactor=0.485 g.

### MRC Ex 15 with SL Ex 7

Repeat MRC Ex 6, but with changes: use SL Ex 7 (80 mg). Of the 40 mL, of anhydrous MeOH add about 18 mL to the vial and mix (solution 1) and use the remaining MeOH with the CH₃I (0.30 mL, 0.5 mol. %) to form solution 2.

While stirring at 600 rpm, pressurize the reactor to 2.76 MPa H₂ (total H₂ added=0.237 g). At 2.41 MPa raise the temperature of the reactor to 140° C. After the temperature reaches 130° C., increase the pressure of the reactor to 6.2 i MPa by adding SynGas 1:1 (vol. H₂:Vol. CO). The actual internal temp was only 138° C. for the first 30 minutes of the reaction. Maintain the pressure at 6.21 MPa by confeeding a mix of SynGas 0.92:1. Stir the reaction for 1 hour at 140° C. At time=15 min, the CO feed rate was 0.080 g/min; at 30 minutes, the rate was 0.052 g/min; at 45 minutes, the rate was 0.037 g/min; at 60 minutes, the rate was 0.028 g/min. After 1 hour, stop the reaction by closing the gas feeds and turning the reactor temperature to below 50° C. Vent the reaction once the temperature drops below 50° C.

Measure the product distribution by GC in dioxane as in MRC Ex 6. Determine yields using TOL as an internal standard: reductive carbonylation products 1,1-Dimethoxyethane=1.97%, acetaldehyde=0.06%, MeOH=85.2%, methyl acetate=0.11%, ARCTF (h⁻¹)=203, mass balance=91.4%. Total CO added to reactor=1.465 g, total H₂ added to reactor=0.339 g.

### MRC Ex 16 with SL Ex 8

Repeat MRC Ex 6, but with changes: use 52 mg of Rh(acac)(CO)₂; use 129 mg of SL Ex 8; of the 40 mL of anhydrous MeOH add about 18 mL to the vial and mix; mix 0.3 mL of CH₃I with the remaining MeOH.

While stirring at 600 rpm, pressurize the reactor to 2.76 MPa H₂ (total H₂ added=0.237 g). At 2.41 MPa raise the temperature of the reactor to 140° C. After the temperature reaches 130° C., increase the pressure of the reactor to 6.21 MPa by adding SynGas 1:1 (vol. H₂:Vol. CO). Maintain the pressure at 6.21 MPa by confeeding a mix of SynGas 0.92:1. Stir the reaction for 1 hour at 140° C. After 1 hour, stop the reaction by closing the gas feeds and turning the reactor temperature to below 40° C. Vent the reaction once the temperature drops below 40° C.

Measure the product distribution by GC in dioxane as in MRC Ex 6. Determine yields using TOL as an internal standard: reductive carbonylation products 1,1-Dimethoxyethane=7.03%, acetaldehyde=1.26%, MeOH=62.6%, methyl acetate=0.97%, ARCTF (h⁻¹) 415, mass balance=86.9%. Total CO added to reactor=3.156 g, total H₂ added to reactor 0.466 g.

### MRC ComEx C with SL dppp

Repeat MRC Ex 6, but with changes: use 100 mg of Rh(acac)(CO)₂ (0.04 mol. %); use dppp (120 mg, 0.03 mol. %).

Measure the product distribution by GC in dioxane as in MRC Ex 6. Determine yields using TOL as an internal standard: ethanol equivalents 1,1-Dimethoxyethane=4.26%, MeOH=76.8%, methyl acetate=1.06%, ARCTF (h⁻¹)=71, mass balance=91.7%. Total CO added to reactor=2.53 g; total H₂ added to reactor=0.36 g.

### MRC ComEx D with SL ComEx B

Repeat MRC Ex 6, but with changes: use 100 mg of Rh(acac)(CO)₂ (0.04 mol. %); use 192 mg of SL ComEx B (0.04 mol. %); add N₂ to pressurize the reactor to 1.38 MPa and vent. Measure the product distribution by GC in dioxane as in MRC Ex 6. Determine yields using TOL as an internal standard: ethanol equivalents 1,1-Dimethoxyethane=6.31%, acetaldehyde=0.43%, MeOH=82.0%, methyl acetate=1.87%, ARCTF (h⁻¹)=88, mass balance=90.7%. Total CO added to reactor=3.12 g; total H₂ added to reactor=0.40 g.

### MRC ComEx E with SL ComEx A

Repeat MRC Ex 6, but with changes: use 52 mg of Rh(acac)(CO)₂ (0.02 mol. %); use 118 mg of SL ComEx A (0.02 mol. %); of the 40 mL of anhydrous MeOH add about 17 mL to the vial and mix; add N₂ to pressurize the reactor to 1.38 MPa and vent. While stirring at 600 rpm, pressurize the reactor to 2.76 MPa H₂ as described in MRC Ex 6. Heat the reactor to 140° C. Once the temperature of the contents of the reactor reaches 135° C., increase reactor pressure to 6.21 MPa with SynGas 1:1 (vol H₂:vol CO). Maintain the pressure at 6.21 MPa with the SynGas (1:1). After 1 hr stop the reaction by closing the mix of SynGas (1:1) and turning the reactor temperature to below 40° C. Vent the reactor.

Measure the product distribution by GC in dioxane as in MRC Ex 6. Determine yields using TOL as an internal standard: reductive carbonylation products 1,1-Dimethoxyethane=4.02%, acetaldehyde=0.24%, MeOH=78.1%, methyl acetate=0.11%, ARCTF (h⁻¹)=213, mass balance=91%. Total CO added to reactor=2.01 g; total H₂ added to reactor=0.38 g.

MRC Ex 6 and MRC Ex 7 each provide a SL with an approximately fourfold increase in turnover frequency as compared to the use of dppp in MRC CompEx C. MRC CompEx D provides a nearly identical selectivity and a similar turnover frequency as the dppp ligand, despite a different backbone flexibility. MRC CompEx E demonstrates that bisphosphine ligands containing para-alkoxy-substituted aryl rings also provide increased turnover frequency as compared to the use of dppp in MRC CompEx C; however, the ortho-alkoxy substituted ligands (Ex 6, Ex 7, Ex 8, 12, 16) are superior SLs for catalysis. Most surprising was that the MRC Ex 12 provided for over a twelve fold increase in selectivity with improved MeOH conversion as compared to the use of dppp in MRC CompEx C.

### Ethanol Reductive Carbonylation Ex 13 with SL Ex 2

Add 0.177 mg Rh(acac)(CO)₂ (0.1 mol. %) and 0.403 mg of SL Ex 2 (0.1 mol. %) to a glass vial in a N₂ purged glovebox. Add TOL (4.1 mL) to the contents of the glass vial and mix. Add EtOH (16 mL) to the contents of the glass vial mix thoroughly to dissolve the Rh(acac)(CO)₂ and SL Ex 2 (solution 1). Add EtOH (24 mL) to CH₃CH₂I (0.55 mL, 1.0 mol. %) to a second vial (solution 2). Take solution 1 up in a first syringe and take solution 2 up in a second syringe. Inject solutions 1 and 2 into a Hastelloy C Parr reactor (“reactor,” with a Hastelloy C bottom valve to drain contents) open to air through a 3.35 mm valve. Close the valve and pressurize the reactor to 1.38 MPa N₂ and vent to remove oxygen; repeat.

While stirring at 600 rpm, pressurize the reactor to 2.76 MPa H₂ as described in MRC Ex 6. Heat the reactor to 140° C. Once the temperature of the contents of the reactor reaches 135° C., to increase reactor pressure to 6.21 MPa with SynGas 1:1 (vol H₂:vol CO). Maintain the pressure at 6.21 MPa with the SynGas (0.92:1). After 2 hr stop the reaction by closing the mix of SynGas (0.92:1) and turning the reactor temperature to below 40° C. Vent the reactor.

Measure the product distribution by GC in dioxane with an FID detector: column temp=35° C. for 2 min, 20° C./min to 250° C.; flow=1.0 mL/min, column=DB-1701, 30 in, 0.32 mm I.D. Assign the GC peaks by comparison with authentic samples. Determine yields by comparison with TOL as an internal standard: reductive carbonylation products propionaldehyde diethyl acetal=0.43%, EtOH=90.8%, ethyl propionate=0.42%, ARCTF (h⁻¹)=2.2. Mass balance (excluding diethylether)=91.7%. Total CO added to the reactor: 1.20 g. Total H₂ added to the reactor: 0.33 g.

What is claimed is:

1. A catalytic system for reductive carbonylation of an alcohol comprising:
a rhodium complex;
an iodide-containing catalyst promoter; and
a supporting phosphorus-containing bidentate ligand for the rhodium complex containing at least one aromatic substituent covalently attached to at least one phosphorus of the supporting phosphorus-containing bidentate ligand, where the at least one aromatic substituent is substituted in an ortho position with an alkoxy substituent or an aryloxy substituent, and where the reductive carbonylation of the alcohol with carbon monoxide gas and hydrogen gas and the iodide-containing catalyst promoter by the catalytic system produces an aldehyde, an acetal, or a combination thereof.

2. The catalytic system of claim 1, where the supporting phosphorus-containing bidentate ligand is a compound of Formula I: 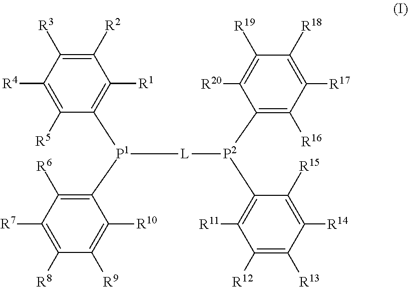
where a linking group, L, includes a chain linking the P¹ and P² atoms of 1 to 10 atoms optionally substituted with R^{v}; where at least one of R¹, R⁵, R⁶, R¹⁰, R¹¹, R¹⁵, R¹⁶ or R²⁰ is of the formula —OR²¹, where R²¹ is a hydrocarbyl group having 1 to 20 carbon atoms, a heterohydrocarbyl group having 1 to 20 atoms each independently selected from C or a heteroatom or a C4 to a C7 cyclic structure that is covalently bound to the aryl group at the meta-position to the respective P¹ and/or P² atom; and R², R³, R⁴, R⁷, R⁸, R⁹, R¹² or R¹³, R¹⁴, R¹⁷, R¹⁸, and R¹⁹ are each independently selected from the group consisting of H, a hydrocarbyl, a heterocarbyl, an aromatic ring, a heteroaromatic ring or a halogen atom.

3. The catalytic system of claim 2, where the linking group, L, is selected from the group consisting of (a) a hydrocarbylene having a chain linking the P¹ and P² atoms of 1 to 4 carbon atoms optionally substituted with R^{v}, (b) a heterohydrocarbylene having a chain linking the P¹ and P² atoms of 1 to 4 atoms each independently a C or a heteroatom optionally substituted with R^{v} and (c) a ferrocenyl group.

4. The catalytic system of claim 3, where L is a hydrocarbylene having 2 or 3 carbon atoms.

5. The catalytic system of claim 2, where the supporting phosphorus-containing bidentate ligand is selected from the group consisting of:

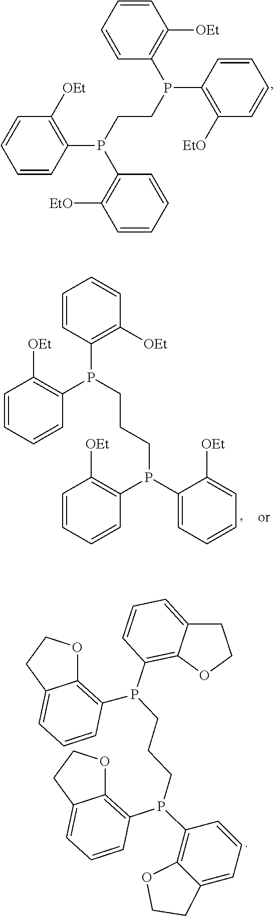 [SMILES: CCOc1ccccc1P(CCP(c1ccccc1OCC)c1ccccc1OCC)c1ccccc1OCC ; CCOc1ccccc1P(CCCP(c1ccccc1OCC)c1ccccc1OCC)c1ccccc1OCC ; c1cc2c(c(P(CCCP(c3cccc4c3OCC4)c3cccc4c3OCC4)c3cccc4c3OCC4)c1)OCC2]

6. The catalytic system of claim 1, where the alcohol is selected from the group consisting of methanol and ethanol.

7. The catalytic system of claim 1, where the iodide-containing catalyst promoter is methyl iodide.

8. A method of methanol homologation to ethanol comprising:
a catalytic system as provided in any one of claims 1 through 7; and
hydrolysis and subsequent hydrogenation of at least a portion of the aldehyde and the acetal of the catalytic system to ethanol.

9. A supporting phosphorus-containing bidentate ligand of Formula I: 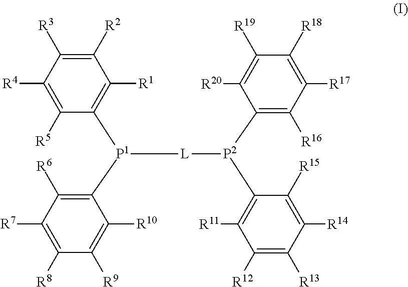
where a linking group, L, includes a chain linking the P¹ and P² atoms of 1 to 10 atoms optionally substituted with R^{v}; where at least one of R¹, R⁵, R⁶, R¹⁰, R¹¹, R¹⁵, R¹⁶ or R²⁰ is of the formula —OR²¹, where R²¹ is a C4 to a C7 cyclic structure that is covalently bound to the aryl group at the meta-position to the respective P¹ and/or P² atom; and R², R³, R⁴, R⁷, R⁸, R⁹, R¹², R¹³, R¹⁴, R¹⁷, R¹⁸, and R¹⁹ are each independently selected from the group consisting of H, a hydrocarbyl, a heterocarbyl, an aromatic ring, a heteroaromatic ring or a halogen atom.

10. The supporting phosphorus-containing bidentate ligand of claim 9, where the supporting phosphorus-containing bidentate ligand has the formula: 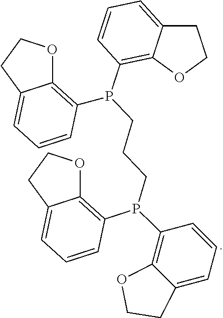 [SMILES: c1cc2c(c(P(CCCP(c3cccc4c3OCC4)c3cccc4c3OCC4)c3cccc4c3OCC4)c1)OCC2]
